(19) United States
(12) Patent Application Publication
(10) Pub. No.: US 2006/0042948 A1
(43) Pub. Date: Mar. 2, 2006

(54) Microfluidic electrophoresis chip having flow-retarding structure

(75) Inventors: Juan G. Santiago, Fremont, CA (US); Byoungsok Jung, Mountain View, CA (US); Rajiv Bharadwaj, Mountain View, CA (US)

Correspondence Address: WOMBLE CARLYLE SANDRIDGE & RICE, PLLC, P.O. BOX 7037, ATLANTA, GA, 30357-0037, US

(73) Assignee: The Board of Trustees of the Leland Stanford Junior University, Palo Alto, CA (US)

(21) Appl. No.: 10/932,078

(22) Filed: Sep. 2, 2004

(51) Int. Cl. C07K 1/26; G01N 27/447

(52) U.S. Cl. 204/450; 204/600

(57) ABSTRACT

A capillary electrophoresis device and separation protocol uses a hydraulic resistance-providing structure (HRPS) in the main separation channel to separate the divide the main separate channel into an upstream portion and a downstream portion. The HRPS may take the form of a porous plug, or a solid plug provided with at least one shallow channel. A sample separates and migrates through the porous structure or the shallow channel, upon application of a voltage difference between the upstream and downstream sides. Among other things, the HRPS helps reduce electrokinetic flow in the presence of conductivity gradients and facilitates robust, high-gradient on-chip field amplified sample stacking. The HRPS also enables the use of a pressure-injection scheme for the introduction of a high conductivity gradient in a separation channel and thereby avoids flow instabilities associated with high conductivity gradient electrokinetics. The approach also allows for the suppression of electroosmotic flow (EOF) and benefits from the associated minimization of sample dispersion caused by non-uniform EOF mobilities. An injection procedure employing a single pressure-flow high-conductivity buffer injection step followed by standard high voltage control of electrophoretic fluxes of sample, may be employed.

### GOVERNMENT RIGHTS

A portion of the work associated with the present invention was funded by DARPA grant F30602-00-2-0609. The U.S. Government may have rights to the present invention.

### FIELD OF THE INVENTION

The present invention is directed to microfluidic devices for carrying out electrophoresis. More particular, the present invention is directed to devices and methods designed for Field Amplified Sample Stacking (FASS) applications and their integration with electrophoretic separations.

### BACKGROUND

On-chip electrophoresis devices offer reduced sample volumes, rapid analysis time, and ease of automation. One drawback of microchannels is that the depth dimensions of etched channels (typically 10-20 μm deep) provide a short line-of-sight-integration length for optical detectors, and this adversely affects their limit of detection (LOD). One way of improving LOD is to integrate an on-line preconcentration process for sample analytes. Sample preconcentration offers higher sensitivity assays, robust electrokinetic injection schemes, and the use of detection modes less sensitive than fluorescence, such as electrochemical detection. Field-amplified sample stacking (FASS) has been used with free-standing capillaries, and also microchips. FASS is one of the most important preconcentration methods for on-chip electrophoresis as it is easily implemented into on-chip capillary zone electrophoresis (CZE) systems and provides a single-step method of achieving high sensitivity. In the past, on-chip FASS, as a stand-alone method, has been limited to less than 10² fold increases in signal strength.

In conventional on-chip FASS systems, a sample analyte is dissolved in a solution of low ionic conductivity, and a small volume of this solution is introduced into the microchannel system using various electrokinetic—or pressure—injection methods. U.S. Pat. No. 6,695,009, whose contents are incorporated by reference to the extent necessary to understand the present invention, shows one prior art approach to sample stacking.

FIGS. 1a & 1b show a schematic of on-chip FASS in the absence of electroosmotic flow (EOF), in a microchip 102 having a “double-T” construction The microchip is provided with first 104a and second 104b regions of high conductivity at opposite ends of the main separation channel and a low conductivity region 106 between the side channels. For the purposes of illustration, only sample ions (typically present in lowest concentration) are shown. First, as seen in FIG. 1a, anionic 108a and cationic 108b sample ions are introduced into the horizontal separation channel within a region of low ionic conductivity. And as seen in FIG. 1b, on application of an electric field, E (indicated by the arrow 110), along the separation channel, sample ions exit the low conductivity/high electric field region and enter the high conductivity/low electric field region. Sample concentration increases as sample ions cross the interface between the high and low conductivity buffers. Cations electromigrate in the direction of electric field and stack at the interface on the cathode side, while anions stack at the anodic interface.

### SUMMARY OF THE INVENTION

In one aspect, the present invention is directed to a capillary electrophoresis microchip having a hydraulic resistance-providing structure (HRPS) in a main separation channel thereof. The HRPS divides the main separation channel into upstream and downstream portions. In one embodiment, the HRPS is a porous polymer plug formed in the main separation channel. In another embodiment, the HRPS is a channeled plug provided with one or more shallow channels.

In another aspect, the present invention is directed to a method of performing electrophoresis using such a microchip. A first buffer having a first conductivity can be introduced into both the upstream and downstream portions of the main separation channel, into the first side channel and into the second side channel. A second buffer having a second conductivity may then be introduced into the upstream portion and the first and second side channels, but not into the downstream portion, first conductivity being higher than the second conductivity. A sample is then introduced into the main separation channel and a separation voltage applied, which causes at least a part of the sample to migrate through said HRPS and into the downstream portion.

In another aspect, the present invention is directed to making such microchips:

In the case of the porous polymer plug, a monomer solution is introduced into main separation channel, a mask applied, and then uncovered portions of the monomer are activated using UV light.

In the case of the channeled plug, the upper surface of the substrate is etched to form the upstream portion, etched to form the downstream portion, and etched to form one or more plug channels in the region between the upstream and downstream portions. The etching may be done in any sequence, including having the upstream and downstream portions etched at the same time. Regardless of the etch sequence, in the resulting device, the one or more plug channels connect the upstream portion with the downstream portion, thereby permitting fluid flow there between. In this embodiment, the channeled plug has unitary, one-piece construction with the substrate.

In an alternate embodiment for forming the channeled plug, a plug is formed as a separate plug insert with bottom and side surfaces that conform to the contour of the main separation channel of a microchip, and an upper surface provided with one or more channels. The separate plug insert is then positioned and fixed in the main separation channel using an adhesive or the like.

In another aspect, the present invention is directed to a method of reducing electrokinetic flow instabilities during electrophoresis of a sample across a conductivity gradient in a main separation channel of a microfluidic electrophoresis chip. The method calls for providing a high hydraulic resistance region in the main separation channel between an upstream portion and a downstream portion, introducing first and second buffers on different sides of the high hydraulic resistance region, introducing a sample into the upstream portion, and then applying a voltage to cause the sample to separate and migrate in the direction of the downstream portion.

In yet another aspect, the present invention is directed to a method of performing electrophoresis on a sample present in a main separation channel of a microfluidic electrophoresis chip. This is done by first providing a high hydraulic resistance region in the main separation channel between an upstream portion and a downstream portion, subjecting the sample to an electric field so as to form a stacked sample on an upstream side of the hydraulic resistance region, applying a voltage difference between the upstream side and a downstream side of the HRPS that is sufficient to cause the stacked sample to separate and migrate through the HRPS; and detecting the sample after it has separated and migrated. In still another aspect, a system in accordance with the present invention employs a simple pressure flow control scheme that uses a single pressure-driven loading step for high conductivity buffer, followed by a single pressure-driven loading step for low conductivity buffer, followed by a single pressure-driven loading step for sample ions. These loading steps are then followed by standard high voltage electrokinetic injection process.

### BRIEF DESCRIPTION OF THE DRAWINGS

FIGS. 1a & 1b illustrate field amplified sample stacking;

FIG. 2a shows a microchip having a hydraulic resistance-providing structure (HRPS) in accordance with the present invention;

FIGS. 2b & 2c show alternate configurations for HRPS in a microchip in accordance with the present invention;

FIG. 3a illustrates a method of introducing oil and monomer into a microchip to create a polymer plug;

FIG. 3b shows a mask covering the substrate to form a polymer plug;

FIG. 3c shows an arrangement for initiating the monomer with light;

FIGS. 4a-4d illustrate a field amplified sample stacking/capillary electrophoresis (FASS/CE) assay protocol using a microchip in accordance with FIG. 2a.

FIG. 5 shows an apparatus for separating and detecting samples;

FIGS. 6a, 6b and 6c show views of a second embodiment of a portion of a microchip in accordance with the present invention;

FIG. 7a shows a section of the main separation channel having an HRPS in the form of an obstruction provided with at least one shallow channel; and

FIGS. 7b & 7c show cross-sections taken along lines of 3b-3b and 3c-3c, respectively, of FIG. 3a.

### DETAILED DESCRIPTION

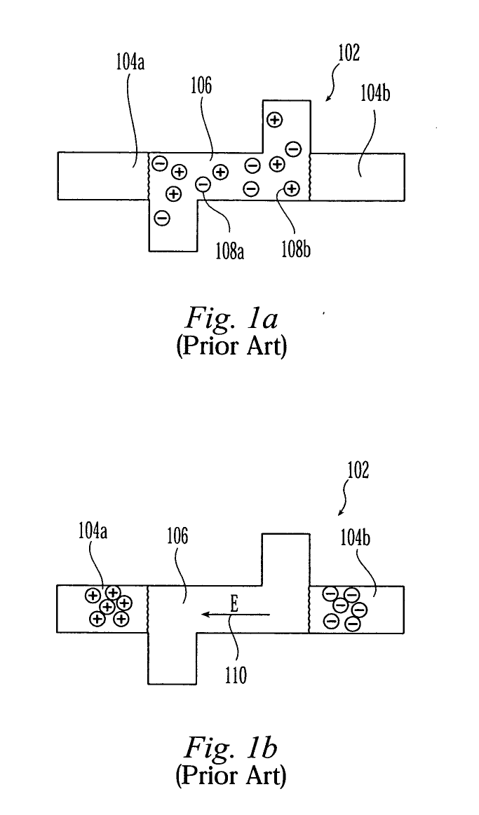

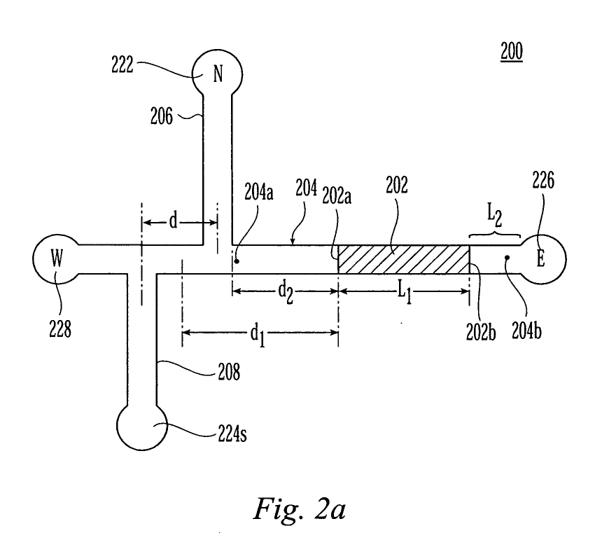

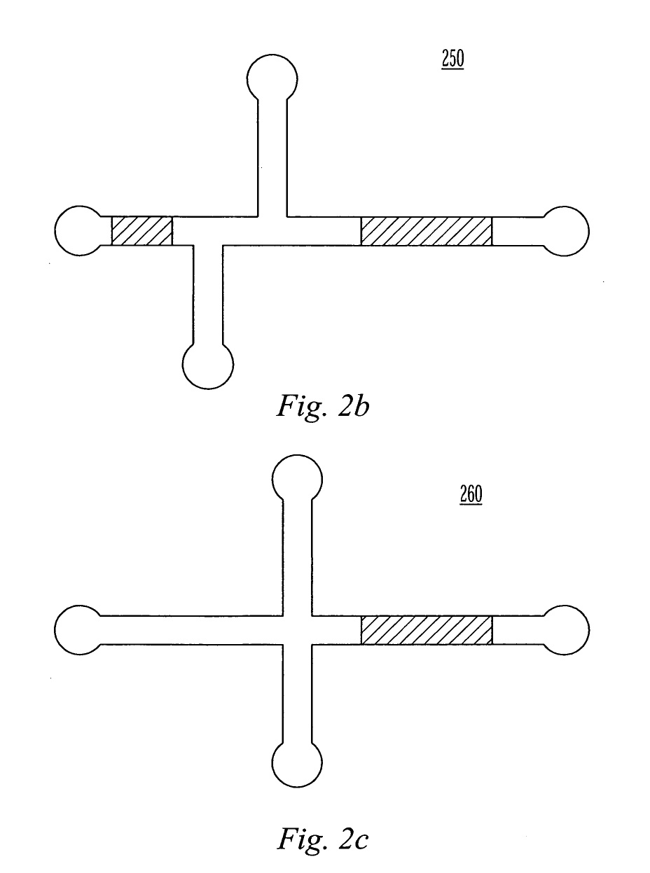

FIG. 2a shows a microchip 200 in accordance with the present invention. The microchip 200 has a hydraulic resistance-providing structure (HRPS) 202 of length L1 along the horizontal, main separation channel 204. The HRPS 202 is positioned such that an ‘open’ channel extends on either side. Thus, the HRPS 202 has an upstream interface 202a facing an upstream portion 204a of the main separation channel 204 and a downstream interface 202b facing a downstream portion 204b of the main separation channel 204. As seen in FIG. 1, the downstream portion 204b extends for some non-zero length L2.

Connected to the main separation channel at a first channel center point is a first, or north, side channel 206. A second, or south, side channel 208 is connected to the main separation channel 204 at a second channel center point. In a preferred embodiment, the first and second channel center points are spaced apart from each other by a distance d and so the microchip has a “double-T” construction.

The ends of the various separation channels are provided with reservoirs 222, 224, 226 and 228 for the introduction of buffers, samples other fluids and materials. In this regard, the first side channel 206 is provided with north reservoir 222; the second side channel 208 is provided with south reservoir 224, and the main separation channel 204 is provided with east reservoir 226 on the downstream side 204b and west reservoir 228 on the upstream side 204a.

The length L1 of the HRPS 202 preferably is between 0.01 mm and 5 mm, more preferably between 0.1 mm and 1.0 mm and most preferably is about 0.5 mm. It is understood, however, that the HRPS 202 may be of some other length, instead. The HRPS is a distance d1 from the center point between the two side channels and a distance d2 from the nearest portion of the closest side channel, which in the construction shown is the first side channel 206. In a preferred embodiment, d1 is between 0.2 mm and 0.4 mm, and more preferably about 0.31 mm.

FIG. 2b shows a double-T microchip 250 with dual HRPS's, one on either side of the side channels, and FIG. 2c shows an X-type microchip 260 with a single HRPS. Other configurations for placement of one or more HRPS's are also possible.

One function of the HRPS is to retard flow between the upstream 204a and downstream 204b portions of the main separation channel 204. In the present invention, the HRPS is implemented in one of two general ways: (1) providing a porous polymer plug in the main separation channel 204; or (2) providing a solid obstruction in the main separation channel, the solid obstruction having at least one shallow channel which connects the upstream 204a and downstream 204b portions of the main separation channel. Both approaches result in a structure that retards or otherwise constricts the flow of liquid between the upstream 204a and downstream 204b portions.

### POROUS POLYMER PLUG AS THE HRPS

The present inventors have described implementation and experimentation of a device in accordance with the present invention having a porous polymer plug in: Jung, B., Bharadwaj, R. & Santiago, J. G., “Thousand-Fold Signal Increase Using Field-Amplified Sample Stacking for On-Chip Electrophoresis”, Electrophoreses 2003, v. 24, No. 19-20, (Oct., 2003). The contents of this paper are incorporated by reference.

Formation of Porous Polymer Plug

The starting point for the polymer plug implementation was a commercially available microchip from Micralyne of Alberta, Canada (www.micralyne.com). The microchip has a double-T geometry, with a channel width of 50 μm and a channel depth everywhere at a maximum 20 μm.

The porous polymer plug was fabricated using a photoinitiated polymerization process similar to that described in Yu, C., Xu, M. C., Svec, F., Frechet, J. M. J., “Preparation of Monolithic Polymers with Controlled Porous Properties for Microfluidic Chip Application”, J. Polymer Science Part A 2002, 40, 755-769, whose contents are incorporated by reference. Ethylene dimethacrylate (EDMA; Sartomer, PA), glycidyl methacrylate (GMA; Sartomer, PA), and azo-bisisobutyronitrile (AIBN; Aldrich, Wis.) were obtained. The monomer (EDMA 0.96 g, GMA 1.421 g), porogenic solvent (50/50 wt % methanol/ethanol 3.6 g), and photoinitiator (AIBN 24 mg) are mixed and then purged with nitrogen for 10 min before use. Prior to introducing the monomer, the microchip was prepared by first rinsing with 0.1 M NaOH for 10 minutes, and then rinsing with deionized water for 30 minutes using a syringe pump.

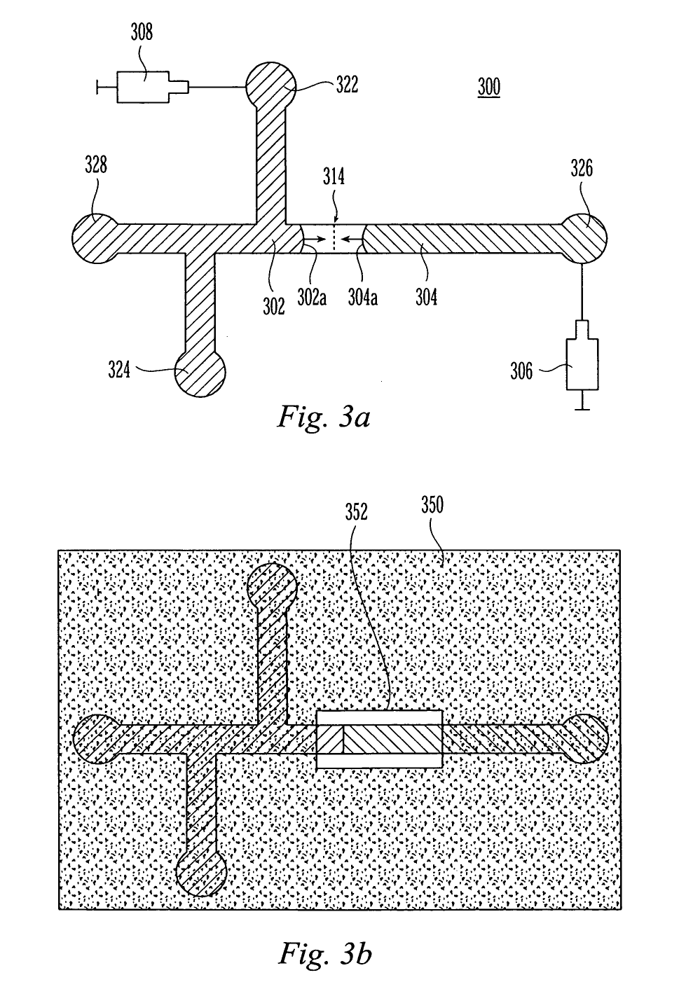

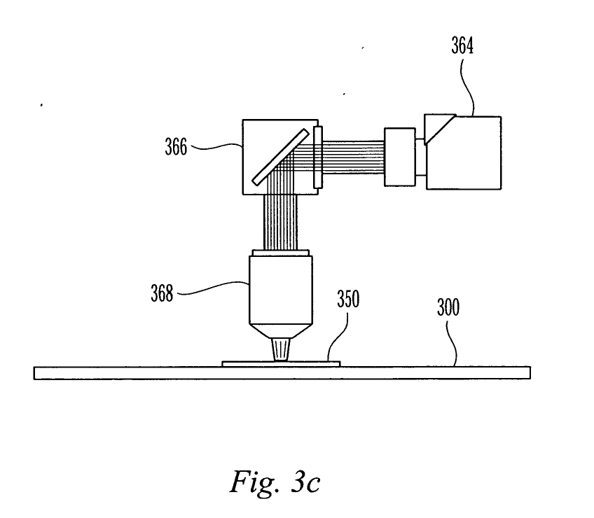

FIGS. 3a -3c show the process for forming a porous polymer plug-type HRPS 102 in accordance with one embodiment of the invention. The upstream interface 104a of the porous polymer plug-type HRPS is defined by an immiscible interface of oil and the monomer solution.

In the microchip 300, monomer solution 304 is introduced into the east reservoir 326 in a controlled manner, such as by a first syringe 306 driven by a first syringe pump under computer control. As the monomer solution 304 is being introduced, oil 302 is simultaneously introduced into the north 322 reservoir, also in a controlled manner, such as by a second syringe 308 driven by a second syringe pump under computer control. It is understood that instead of, or in addition to, the north reservoir 322, the oil may be introduced into the south 324 and/or west 328 reservoirs, as well. Regardless of into which reservoir(s) the oil 302 is introduced, one may control the rates of introduction of the monomer 304 and the oil 302 such that the leading oil front 302a and the leading monomer front 304a move toward each other as indicated by the arrows in FIG. 3a, and ultimately meet at the future upstream interface 314. The immiscibility of oil and the monomer helps ensure that the boundary between them is well-defined.

After the oil 302 and monomer 304 have been loaded into the channels and have met at the future upstream interface 314, a mask 350 having a window 352 is placed over the microchip 300. In a preferred embodiment, the mask 350 is a printed ink-on-mylar film shadow mask, and the window 352 permits exposure of only that portion of the monomer 304 to be polymerized into the porous polymer plug-type HRPS.

As seen in FIG. 3c, broadband light from a mercury arc lamp 364 is focused on the plane of microchip 300 via a UV transmitting filter cube 366 and an epifluorescent microscope 368. In one embodiment, the microchip 300 is exposed for four hours, although other lengths of time may also be used. After the monomer has been photo-polymerized, the remaining monomer is removed from the system, preferably by rinsing the microchannel with methanol for 2 hours and then deionized water for 3 hours using a syringe pump.

In the foregoing photo-polymerization example, due to blurring that results from using the broadband mercury arc lamp 364, an oil-monomer interface was used to provide the porous polymer plug-type HRPS 202 with a more precise upstream interface 202a where the sample for separation is to be introduced, the downstream interface 202b not being as critical. In an alternate embodiment for forming the plug, one may use a laser instead of the mercury arc lamp 364 as the light source. In such an alternate embodiment, the monomer may be introduced throughout the length of the main separation channel, a mask placed over the microchip, and a laser used to perform the photo-polymerization, thereby dispensing with the need to first form the oil-monomer interface. Other methods may also be used to form the polymer plug-type HRPS 202.

The pore diameter distribution of the porous polymer structure can be analyzed by polymerizing monoliths off-chip. In one experiment, a small glass chamber was filled with the same monomer solution, and then exposed to similar polymerization conditions. After polymerization, the monoliths were removed from the glass chamber, washed with methanol and dried. The median pore diameter is about 4.6 μm, with at least 90% of the pores having a diameter between 1 nm and 10 μm. A void volume of the material is about 0.5, but preparations having void volumes on the order of between 0.05 and 0.9 can be prepared.

Buffer & Sample

A low conductivity buffer, a high conductivity buffer and a fluorescent sample are first prepared. A 5 mM HEPES (Sigma, Mo.) buffer solution with a pH of 7.0 was used with a 0.4 wt % methyl cellulose (Aldrich, Wis.) solute to suppress electroosmotic flow (EOF). This serves as the “low conductivity buffer”. A high conductivity buffer (77.6 mS/cm) was prepared by dissolving a requisite amount of NaCl salt (J. T. Baker, N.J.) to the HEPES buffer. The sample solute comprises an aqueous solution of 1 μM bodipy dye (available from Molecular Probes, Oreg.) and 2 μM fluorescein dye (available from J. T. Baker, N.J.). All sample and buffer solutions were filtered with 0.2 μm syringe filter before use. The conductivity of buffer and sample solution were measured using a conductivity meter (available from Jenco Instruments, Calif.).

Field Amplified Sample Stacking/Capillary Electrophoresis (FASS/CE) Assay Protocol

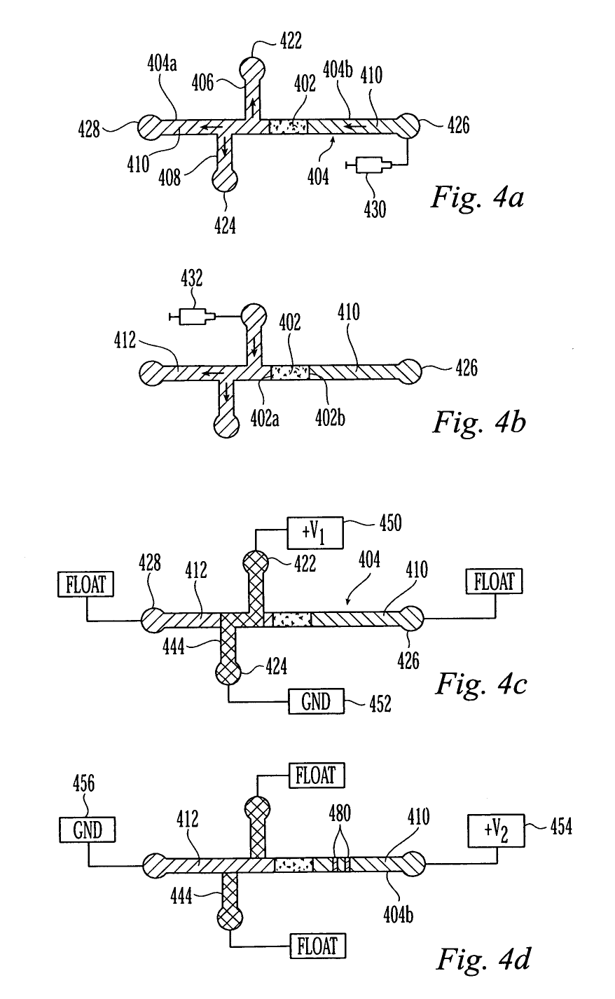

FIGS. 4a-4d illustrate a preferred embodiment of a FASS/CE assay protocol in accordance with the present invention, in which a porous polymer plug-type HRPS 402 was used.

Prior to introducing any buffer, a microchannel glass surface treatment was performed. This was done by rinsing the microchip with a dynamic coating reagent. Although a variety of coating reagents may be employed, the aforementioned 0.4% methyl cellulose solution was used in this role, and so was introduced into the entire microchip by flowing for 30 min. All buffers used in the experiment contain the same amount of methyl cellulose, to help suppress EOF throughout the microchip.

As depicted in FIG. 4a, first, high conductivity buffer 410 is introduced, via the east reservoir 426, into the downstream portion 404b of the main separation channel, through the HRPS, and into the upstream portion 404a, and the side channels 406, 408, as indicated by the arrows. In one embodiment, the high conductivity buffer 410 is introduced by injection with a computer-controlled syringe pump system 430. The syringe pump introduced the high conductivity buffer at a flow rate of about 1.0 μl/min for approximately 1.0 minute. During this introduction, the porous polymer plug-type HRPS provides high hydraulic resistance to buffer flow.

The hydraulic resistance per unit length can be quantified as the ratio of the local pressure gradient to the volume flow rate. A typical 50 micron wide by 20 micron deep channel has a hydraulic resistance per unit length of 4.41×10¹⁶ Pa·s/m⁴. For an exemplary chip in accordance with the present invention, the porous region has a hydraulic resistance per unit length that is roughly 25 times larger, about 1.18×1018 Pa·s/m⁴, based on the equation:

$$R = \frac{Δ P}{QL} = \frac{8 τμ}{ψ {Aa}^{2}} \qquad ( Eq . 1 )$$

where ΔP/L is pressure gradient; Q is flow rate; L is the length of the porous plug; porosity ψ=0.45; A is the cross-sectional area of the porous plug, the average pore diameter a=4.9 μm, tortuosity τ=1.45; the viscosity of the buffer μ=0.001 Pa·s, and assuming no electric field present.

As depicted in FIG. 4b, low conductivity buffer 412 is then introduced from the north reservoir 422 using syringe 432. It is understood, however, that the low conductivity buffer could be introduced via the south 424 or west 428 reservoirs instead. In one embodiment, the low conductivity buffer 412 is introduced at a flow rate of about 0.1 μl/min for 0.5 min. Introducing the low conductivity buffer 412 at a lower pressure and for a lower time than that used to introduce the high conductivity buffer, helps reduce the amount of low conductivity buffer that passes through the porous polymer plug-type HRPS 402 from the upstream interface 402a to the downstream interface 402b. It therefore helps prevent mixing of the low conductivity buffer with the high conductivity buffer in the downstream portion 404b of the main separation channel. In this instance, the porous polymer plug-type HRPS provides high hydraulic resistance which minimizes the mixing of two buffers at the upstream HRPS/buffer interface 402a, as well. The result of this step is that high conductivity buffer 410 occupies the downstream portion 404b of the main separation channel 404 while low conductivity buffer 412 is present in the upstream portion 404a of the main separation channel 404 and also in the first 406 and second 408 side channels.

Next, as seen in FIG. 4c, an anionic sample 444 was then electrokinetically introduced into the double-T injector, via the south reservoir 424. For this, the south reservoir was filled with the sample mixture of bodipy and fluorescein and electrically grounded. A positive voltage source 450 providing a voltage V1, which in one embodiment is 1 kV, was applied to the north reservoir 422 with the south reservoir 424 connected to ground 452 and the east 426 and west 428 reservoirs allowed to electrically float. This creates an electric field that caused negatively charged sample ions to electromigrate from the south reservoir 424 towards the north reservoir 422, with at least a portion of the sample ending up in the main separation channel 404, between the two side channels 406, 408.

Finally, as seen in FIG. 4d, a positive voltage source 454, providing in one embodiment, 3 kV, is applied at the east reservoir 426 while the west reservoir 428 is connected to ground 456, thereby establishing an east-to-west electric field. This field initiates both sample stacking and electrophoretic separation of the negatively charged sample ions. The sample in the main separation channel 404 thus undergoes stacking and migration in the downstream direction through the porous polymer plug-type HRPS 402, and separates into bands 480 which can then be detected in a manner known to those skilled in the art. Preferably, these bands are detected in the downstream portion 404b, as seen in FIG. 4d, though the detection may also be performed while the bands are transiting through the HRPS 402. In a preferred embodiment, the separated sample peaks were detected using an epifluorescent microscope and a CCD camera with a viewing region positioned 10 mm downstream of the injection region.

While specific values are presented in the foregoing description, it is understood that a wide variety of values may be used.

For example, it is understood that the terms “low hydraulic resistance” and “high hydraulic resistance” are relative terms. In general, a “high hydraulic resistance” may be anywhere from 1×10¹⁶ Pa·s/m⁴ to 1×10¹⁹ Pa·s/m⁴, depending on the hydraulic resistance of the channel where no plug is present. In general, however, the region of high hydraulic resistance preferably has a hydraulic resistance that is 10-100 times as great as the low hydraulic resistance region.

Furthermore, the terms “low conductivity” and “high conductivity”, as applied to buffers, are relative terms. Thus, a low conductivity buffer may have a conductivity between 1 uS/cm and 1 mS/cm, while a high conductivity buffer has a conductivity that is about 10-10,000 times higher.

As to the voltage applied to effect stacking and separation, it is possible to have this depend on the length of the high hydraulic resistance region. Thus, for instance, one may apply a voltage difference of between 100-100,000 volts, if the length of the high hydraulic resistance region is between 1 and 100 cm, and a voltage difference of between 1-100 volts, if the length of the high hydraulic resistance region is between 0.05 and 1 cm. Preferably, though, the applied voltage is sufficient to cause the sample to enter a region adjacent to the upstream side of the porous plug at a rate between 1 and 100 nl/min.

Detection System

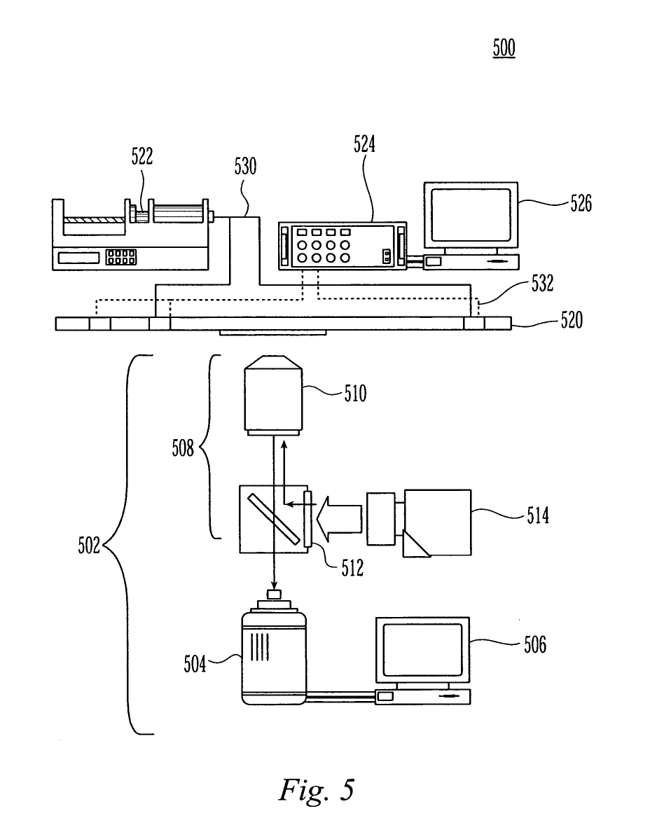

FIG. 5 shows a schematic of an experimental FASS/CE microchip setup 500. The detection/visualization system 502 includes an intensified CCD camera 504 (Roper Scientific, IPentaMAX, N.J.) connected to a computer 506 for processing and display. The CCD camera 504 receives light from an inverted epifluorescent microscope 508 (Olympus, IX70, N.Y.) comprising a 10× objective 510 (numerical aperture (N.A.) of 0.3, Olympus, N.Y.) and a XF100-3 filter cube 512 (Omega Optical, Vt.) with peak excitation and emission wavelength ranges of 450-500 nm and 500-575 nm. A mercury lamp 514, whose beam is directed via the filter cube 512 before impinging on the separated samples, is used to cause the dyes to fluoresce. The setup 500 also includes the microchip 520 itself, a multi-valve syringe pump 522 (Harvard Apparatus, Pump 33, Mass.), for pressure-injection control, and a multi-port high voltage power supply 524 (Micralyne, Alberta, Canada). The syringe pump 522 and the power supply 524 are under the control of computer 526. Various pressure/flow and electrical connections to the microchip are shown as solid 530 and dashed 532 lines, respectively, and are known to those skilled in the art.

### CHANNELED PLUG AS THE HRPS

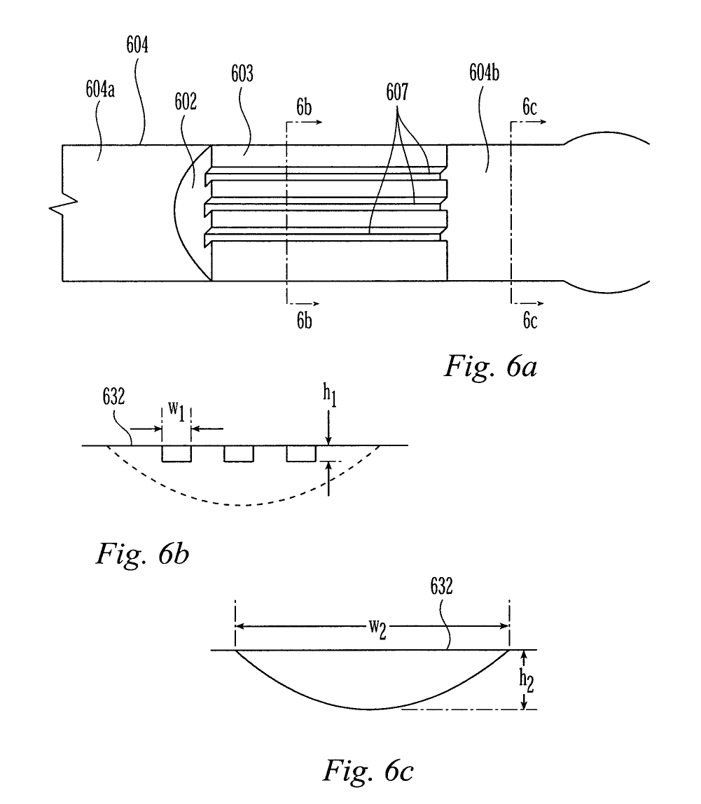

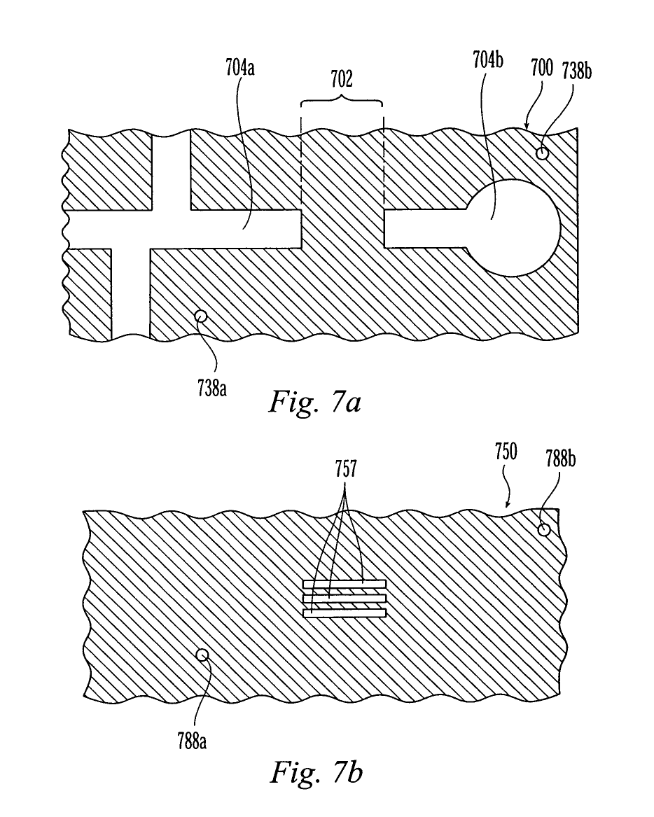

FIG. 6a shows a channeled plug 602 having an upper surface 603 provided with three linear, shallow plug channels 607. The channeled plug 602 preferably is solid in that buffers and the like do not normally pass through the plug material itself, but rather only through the channels 607. Thus, the channeled plug 602 is relatively non-porous, in contrast to the porous polymer plug 402 discussed above. Preferably, the channeled plug 602 is formed of the same material as the substrate in which the main separation channel is formed.

It is understood that the upper surface 603 of the channeled plug 602, as well as the rest of the main separation channel 604, are under a glass surface 632, as is typical with microchips. It is also understood that a different number, such as 1, 2, 4 or even more, plug channels may be provided. It is further understood that the plug channels do not necessarily have to be linear or have the same cross-sectional area, though both are preferable.

The plug channels 607 connect the upstream side 604a of a main separation channel 604 with the downstream side 604b. The plug channels 607 are configured and dimensioned to permit a fluid to pass between the upstream 604a and downstream 604b portions of the main separation channel 604. During the pressure injection protocol, the smaller cross-sectional area of the plug channels 607, relative to that of the main separation channel 604, provides hydraulic resistance to fluid flow. Detection of a migrating sample can take place while the sample still occupies channels 607, or after the sample has exited the channels 607.

The plug channels 607 have a plug channel depth h1 that is less than a depth h2 of the main separation channel. The plug channel depth h1 is nominally between 100 nm and 2 μm although it may take on other heights, as well. Furthermore, the plug channel depth h1 preferably is no greater than 1/10 the depth h2 of the main separation channel. The plug channels have a plug channel width w1 that is less than a width w2 of the main separation channel. The plug channel width w1 is nominally between 1 μm and 10 μm. Furthermore, the plug channel width w1 is no greater than ⅕ the width w2 of the main separation channel. And while the channels 607 formed in the upper surface of the plug 603 preferably have a rectangular cross-section, they may instead take on other cross-sectional shapes.

In one embodiment, the plug has unitary one-piece construction with the substrate. In such case, the channels 607 and the upstream and downstream portions are formed of one continuous piece of substrate material, and the substrate is subjected to etching and/or machining to create the various formations therein.

In an all-etch process, a first portion of the substrate is etched to form an upstream portion of the main separation channel, a second portion of the substrate is etched to form a downstream portion of the main separation channel, and one or more shallow channels are etched in a third portion of the substrate, the one or more shallow channels in the resulting structure connecting the upstream and downstream portions. The various etching is performed under appropriate conditions so that the etched shallow channel depth h1 is less than a depth h2 of either the upstream portion or the downstream portion. Preferably, the upstream and downstream channels are etched simultaneously, and then the shallow channels are etched. However, the present invention contemplates that these three portions of the substrate can be etched in any order in either two or three separate steps.

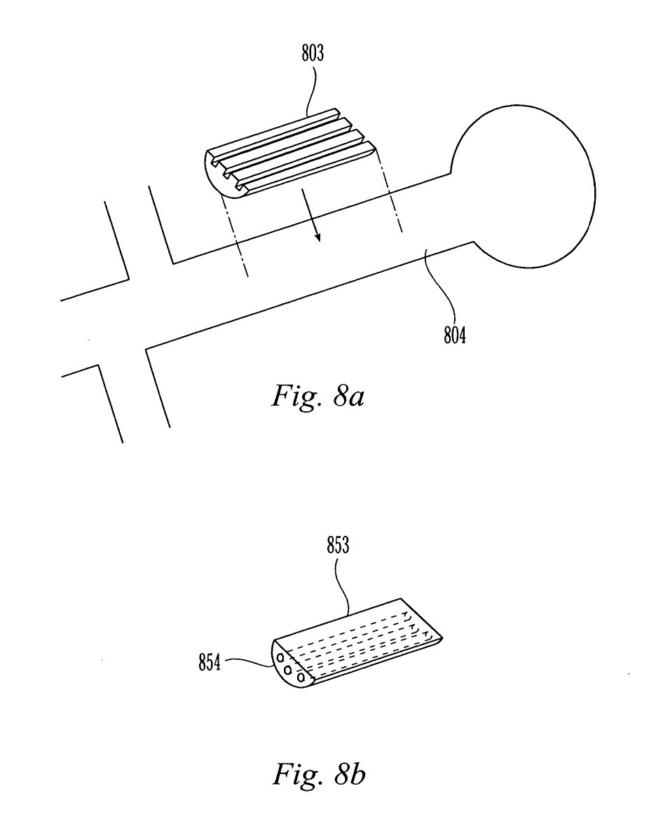

FIG. 7a shows an example of a mask 700 that can be used to prepare for simultaneously etching both the upstream and downstream portions of a main separation channel. The mask 700 has a first opening 704a that corresponds to the region where at least the upstream portion will be formed and a second opening 704b that corresponds to the region where at least the downstream portion will be formed. The mask 700 has a channel portion 702 that separates the first 704a and second 704b openings. The mask 700 also has a pair of alignment marks 738a, 738b to facilitate positioning the openings in the proper locations.

FIG. 7b shows an example of a mask 750 that can be used to prepare for etching the channels 607 of the channeled plug 602. The mask 750 has a plurality of slots 757 that correspond to the positions where the channels 607 are to be formed. The mask 750 also has a pair of alignment marks 788a, 788b that match the location of alignment marks on mask 700. This results in the main separation channel having an elevated portion provided with the plug channels

Preferably, mask 700 is used to etch the upstream 604a and downstream 604b portions in a first etching step, and then mask 750 is used to etch the channels 607 in a second etching step.

FIG. 8a depicts an alternative embodiment for preparing a microchip in accordance with the present invention, a plug insert 803 is first formed. The plug insert 803 has a lower surface that conforms to the cross-sectional, typically D-shaped, contour of the main separation channel 804 of a microchip. The upper surface of the plug insert 803 is provided with one or more channels, whose shape and dimensions are described above, the channels being formed by etching or machining. Regardless of how it is formed, as depicted by the arrow in FIG. 8a, the plug insert 803 ultimately is placed in the main separation channel 803 and fixed thereto by means of an adhesive or the like.

As seen in FIG. 8b, an alternative plug insert 853 has plurality of channels 854 formed within, and along, the body of the insert 853 in a longitudinal direction. In such case, during the pressure injection protocol, the buffers and other materials pass though the body of the plug insert 854, and sample detection occurs only after the sample has exited the plug insert 854 on the downstream side of the main separation channel.

From the foregoing, it is evident that the term ‘plug’, as used herein, covers a structure that (a) is formed, in situ, in a main separation channel (such as the porous polymer plug), (b) is formed as a separate component, and then inserted into the main separation channel (such as the plug insert), or (c) has unitary construction with the main separation channel (such as being formed by etching a region of the substrate located between what are, or will become, the upstream and downstream sides).

It is further understood that one uses the channeled plug-type HRPS in a manner similar to that of the porous polymer plug-type HRPS, described above. Thus, a substantially similar pressure-injection protocol may be employed with channeled plug-type HRPS. Generally speaking, the HRPS 202, however implemented, provides a region of high hydraulic resistance to pressure driven flow that still allows electrophoretic migration to take place. The above-described pressure-injection protocol takes advantage of this, resulting in two consequences.

First, the pressure-injection protocol results in a device having a high conductivity gradient within the separation channel while still having suppressed electroosmotic flow, EOF suppression being realized in the above-described embodiment by the use of methyl cellulose. Suppressing the EOF helps reduce sample dispersion during the simultaneous FASS/CE process.

Second, the pressure-injection protocol helps reduce electrokinetic instabilities. As is known to those skilled in the art, electrokinetic instabilities are associated with high conductivity gradient regions near channel intersections where conductivity gradients and electric fields are three-dimensional. Such electrokinetic instabilities can cause excessive dispersion of the buffer-buffer interface, thereby limiting the performance of FASS with high stacking ratios. The pressure-injection scheme allows for the establishment of an initial conductivity gradient within the separation channel, followed by sample introduction into one side channel, and application of a voltage V1 across both two side channels, thereby creating an electric field and causing the sample to enter into the main separation channel. In particular, the protocol allows for a voltage V1 creates an electric field sufficiently large to introduce a portion of the sample into the main separation channel, yet not so large as to induce electrokinetic instabilities at the upstream interface 402a of the HRPS 402.

Finally, while the present invention has been described with respect to one or more preferred embodiments, it should be kept in mind that variations from this are also contemplated to be within the scope of the invention, as claimed below.

What is claimed is:

1. A microfluidic electrophoresis chip comprising:
a main separation channel having a first hydraulic resistance-providing structure (HRPS) that divides the main separation channel into an upstream portion and a downstream portion;
a first side channel connected to the main separation channel at a first point on the upstream portion and on a first side thereof; and
a second side channel connected to the main separation channel at a second point on the upstream portion, and on a second side thereof;

2. The chip according to claim 1, wherein the second point is between the first point and the HRPS, such that the first and second side channels have a double-T structure.

3. The chip according to claim 1, wherein the first point and the second point are co-located such that the first and second side channels form a single continuous channel that crosses the main separation channel.

4. The chip according to claim 1, further comprising a second HRPS positioned in the upstream portion of the main separation channel such that the first and second points are between the first and second HRPS.

5. The chip according to claim 1, wherein the first HRPS has a length between 0.01 mm and 5 mm.

6. The chip according to claim 1, wherein the first HRPS comprises a solid plug provided with at least one plug channel configured and dimensioned to permit a fluid to pass between the upstream and downstream portions of the main separation channel.

7. The chip according to claim 6, wherein the solid plug is formed of a same material as a substrate of the chip.

8. The chip according to claim 7, wherein the solid plug has unitary one-piece construction with the substrate.

9. The chip according to claim 6, wherein the first HRPS comprises a plurality of plug channels.

10. The chip according to claim 6, wherein:
the at least one plug channel has a plug channel depth h1 that is less than a depth h2 of the main separation channel.

11. The chip according to claim 10, wherein:
the plug channel depth h1 is between 100 nm and 2 μm.

12. The chip according to claim 10, wherein:
the plug channel depth h1 is no greater than 1/10 the depth h2 of the main separation channel.

13. The chip according to claim 10, wherein:
the at least one plug channel has a plug channel width w1 that is less than a width w2 of the main separation channel.

14. The chip according to claim 13, wherein:
the plug channel width w1 is between 1 μm and 10 μm.

15. The chip according to claim 13, wherein:
the plug channel width w1 is no greater than ⅕ the width w2 of the main separation channel.

16. The chip according to claim 1, wherein the first HRPS comprises a first porous plug positioned in the main separation channel.

17. The chip according to claim 16, wherein the plug comprises a porous polymer.

18. The chip according to claim 16, wherein the plug comprises a porous dielectric material.

19. The chip according to claim 16, wherein the plug comprises a porous bed of packed particulate matter.

20. The chip according to claim 16, wherein the plug is between 0.01 and 10.0 mm in length.

21. The chip according to claim 16, wherein at least 90% of the pores in the plug have a diameter between 1 nm and 10 μm.

22. The chip according to claim 16, wherein a void volume of the plug is between 0.05 and 0.9.

23. A method of conducting electrophoresis comprising:
providing a microfluidic electrophoresis chip comprising:
a main separation channel having a first hydraulic resistance-providing structure (HRPS) that divides the main separation channel into an upstream portion and a downstream portion;
a first side channel connected to the main separation channel at a first point on the upstream portion and on a first side thereof; and
a second side channel connected to the main separation channel at a second point on the upstream portion, and on a second side thereof;
introducing a first buffer having a first conductivity into both the upstream and downstream portions of the main separation channel, into the first side channel and into the second side channel;
introducing a second buffer having a second conductivity into the upstream portion and the first and second side channels, but not into the downstream portion, wherein the first conductivity is higher than the second conductivity;
introducing a sample into the upstream portion of the main separation channel; and
applying a first voltage difference between the upstream portion and the downstream portion to thereby cause at least a part of the sample to migrate through said HRPS and into said downstream portion.

24. The method according to claim 23, wherein the first buffer is first introduced into the downstream portion under pressure such that it passes through the HRPS and enters into the upstream portion and the first and second side channels.

25. The method according to claim 23, wherein said step of introducing a sample into the main separation channel comprises:
introducing the sample at the same time as a portion of the second buffer is introduced; and
applying a first pressure difference across the two side channels to cause buffer-containing sample to migrate from one side channel to the other such that at least a portion of the sample ends up in said main separation channel

26. The method according to claim 23, wherein:
the second point is between the first point and the HRPS, such that the first and second side channels have a double-T structure; and
the second buffer is first introduced into one of the two side channels, the second buffer then entering the upstream portion and the other of the two side channels.

27. The method according to claim 23, wherein:
the second point is between the first point and the HRPS, such that the first and second side channels have a double-T structure; and
said step of introducing a sample into the main separation channel comprises introducing the sample into one of the two side channels and applying a first voltage difference across portions of the two side channels to cause the sample to migrate from one side channel to the other such that at least a portion of the sample ends up in said main separation channel.

28. The method according to claim 23, comprising:
introducing the sample into a region adjacent to the porous plug at a rate between 1 and 100 nl/min.

29. The method according to claim 23, wherein:
the second sample buffer has a conductivity between 1 uS/cm and 1 mS/cm.

30. The method according to claim 23, comprising:
applying a first voltage difference of between 100-100,000 volts, if the length of the HRPS is between 1 and 100 cm.

31. The method according to claim 23, comprising:
applying a first voltage difference of between 1-100 volts, if the length of the HRPS is between 0.05 and 1 cm.

32. A method of forming a porous polymer plug in a predetermined portion of a main separation channel of a microfluidic electrophoresis chip, comprising:
introducing a first material into a first portion of the channel;
introducing a monomer solution including at least one monomer and photoinitiator into a second portion of the channel, the first portion being adjacent to the second portion and the first material being selected such that it is substantially immiscible with the monomer solution;
illuminating only a predetermined section of the second portion of the channel to thereby activate a corresponding section of said monomer solution and form said plug;
removing said first material; and
removing unactivated monomer remaining in said second portion.

33. A method of forming a porous polymer plug in a predetermined portion of a main separation channel of a microfluidic electrophoresis chip, comprising:
introducing a monomer solution including at least one monomer and a photoinitiator into at least said predetermined portion;
providing a mask that exposes said predetermined portion of the main separation channel and covers portions of the main separation channel on either side of said predetermined portion; and
activating the monomer solution with light; and
removing unactivated monomer solution remaining in said main separation channel

34. A method of forming a separation channel in a substrate of an electrophoresis microchip, comprising:
etching a first portion of the substrate to form an upstream portion of the separation channel;
etching a second portion of the substrate to form a downstream portion of the separation channel; and
etching at least one shallow channel in a third portion of the substrate, the at least one shallow channel having a shallow channel depth h1;
such that the at least one shallow channel connects the upstream and downstream portions and the shallow channel depth h1 is less than a depth of either the upstream portion or the downstream portion.

35. A method according to claim 34, comprising simultaneously etching the first and second portions before etching the third portion.

36. A method according to claim 35, comprising etching the third portion before etching either the first or second portion.

37. A method of forming an electrophoresis microchip having a hydraulic resistance-providing structure (HRPS), comprising:
providing a microchip having a separation channel with depth h2;
providing a channeled plug insert having at least one plug channel formed along an upper surface thereof, the at least one plug channel having a plug channel depth h1 which is less than depth h2; and
placing the channeled plug insert in the separation channel such that the at least one plug channel provides a path for passage of a fluid between portions of the separation channel on either side of the channeled plug insert.

38. A method of reducing electrokinetic flow instabilities during electrophoresis of a sample across a conductivity gradient in a main separation channel of a microfluidic electrophoresis chip, the method comprising:
providing a high hydraulic resistance region in the main separation channel between an upstream portion and a downstream portion thereof;
introducing a first buffer having a first conductivity into the upstream portion;
introducing a second buffer having a second conductivity into the downstream portion; and
applying a voltage difference between the upstream portion and the downstream portion to thereby cause at least a part of the sample to migrate from the upstream portion, through the high hydraulic resistance region, and into said downstream portion.

39. The method according to claim 38, comprising providing a high hydraulic resistance region having a hydraulic resistance between 1×10¹⁶ Pa·s/m⁴ and 1×10¹⁹ Pa·s/m⁴.

40. The method according to claim 38, wherein the first and second buffers have different temperatures.

41. The method according to claim 38, wherein the first and second buffers are at the same temperature but have different conductivities.

42. The method according to claim 38, wherein the first and second buffers are at the same temperature but have different viscosities and/or different conductivities.

43. The method according to claim 38, wherein the first and second buffers are at the same temperature but have different pH and/or different conductivities and/or different viscosities.

44. A method of performing electrophoresis on a sample present in a main separation channel of a microfluidic electrophoresis chip, comprising:
providing a high hydraulic resistance region in the main separation channel between an upstream portion and a downstream portion thereof;
subjecting the sample to an electric field so as to form a stacked sample on an upstream side of the hydraulic resistance region;
applying a first voltage difference between the upstream side and a downstream side of the HRPS that is sufficient to cause the stacked sample to separate and migrate through the HRPS; and
detecting the sample after it has separated and migrated.

45. The method according to claim 44, comprising detecting the sample after it has exited the HRPS.

46. The method according to claim 44, comprising detecting the sample while it is still in the HRPS.
